(19) United States
(12) Patent Application Publication
(10) Pub. No.: US 2012/0330204 A1
(43) Pub. Date: Dec. 27, 2012

(54) Therapeutic Cushioning and Devices for Assisting Respiration of and administering fluid to a patient

(75) Inventors: Mary Baldauf, Valley Stream, NY (US); Sonia Chaudhry, Valley Stream, NY (US); Thomas Baldauf, Merrick, NY (US)

(73) Assignee: BALDY BY DESIGN, LLC., Valley Stream, NY (US)

(21) Appl. No.: 13/536,012

(22) Filed: Jun. 28, 2012

Related U.S. Application Data
(63) Continuation-in-part of application No. 12/083,024, filed on Sep. 15, 2009, now abandoned.
(60) Provisional application No. 60/723,079, filed on Oct. 3, 2005.

(51) Int. Cl. A61F 5/055

(52) U.S. Cl. 602/18

(57) ABSTRACT

The present invention is directed to both therapeutic cushioning designed to support the head of a patient in bed to improve respiration and a series of devices designed to assist a patient's respiration and administer fluid to the patient, by ensuring such devices cannot be inadvertently removed from the patient in the absence of medical personnel and cause unwanted injury to the patient. At the same, time, a device is provided to assist a patient's breathing in the absence of such assistive devices, e.g., during sleep.

### BACKGROUND OF THE INVENTION

The present invention relates to cushioning that can be used to prevent skin breakdown or bedsores of a patient. When a patient is immobilized for an extended period of time, pressure can build up in capillaries obstructing or occluding blood flow therethrough, resulting in bedsores. The present invention, which includes variously contoured and/or gel-filled cushions, can be used in a variety of settings to reduce skin irritation and prevent decubitus ulcerations.

The present invention is also directed to a series of devices designed to assist a patient during respiration and/or invasive administering of fluid. More particularly, the present invention is directed to a series of devices designed to immobilize medical or respirational equipment inserted into a patient to avoid unwanted injury to the patient while, at the same time, ensuring effective medical treatment and/or respiration of the patient.

Traditionally, preventing skin breakdown or bedsores has been an enormous task, and is worsening due to the aging population of this country. Specialized beds and mattresses have been employed in an attempt to eliminate this problem, with even rolled up towels being utilized. Bedridden patients must be turned and re-positioned several times a day to avoid bedsore occurrence, with need for closely monitoring a patient's hydration and nutrition status being required. Such intensive monitoring is expensive, time-consuming, and not often effective. Attempts have also been made to apply specialized adhesive dressings such as Moleskin, Duoderm and Tegaderm, to specific areas on the patient.

Thus, the cost of treating such skin breakdown is tremendous and cuts into emphasis on other medical treatment for a patient. The effect is especially debilitating on immuno-suppressed patients who often require extended healing periods. Additionally, emotional toll results from such unwanted occurrences during medical treatment. Once bedsores develop, treatment usually involves surgical debridement. Accordingly, there is a tremendous need to reduce or totally eliminate occurrence of such skin breakdown.

According to the National Decubitus Foundation, over one million hospital patients are subjected to the affliction of bedsores every year. Most victims are in their seventies and eighties. Thus an aging population exacerbates the problem. However, any person who is bedbound or essentially immobile is at great risk for developing decubitus ulcers.

Institutions such as hospitals and nursing homes often use specialized beds, various types of mattresses and even rolled towels in an attempt to prevent skin breakdown. Some institutions have mandates on how often patients must be turned and positioned. They pay close attention to each patient's nutrition and hydration status. However, this is not always possible for all patients. Many treatment teams have devised their own informal system of positioning patients on pillows, using specialized beds or applying Moleskin, Duoderm, Tegaderm or other adhesive dressings to specific areas in an effort to prevent breakdown.

The cost of treating skin breakdown is tremendous. The National Decubitus Foundation reports that the cost of bedsores in hospitals is conservatively 55 billion dollars per year. The scope of the problem is even greater when bedsores in nursing homes and home care are included. The financial toll is staggering—billions of dollars in healthcare costs could be avoided through education and a proper application of resources. For patients who are immuno-suppressed, healing times can be extended. The emotional, psychological and financial burdens for these patients, their families, the insurance companies, and the institutions caring for them is rising each year.

Regarding assisting of respiration, it is also well-known that several invasive devices, e.g., intravenous tubing, assistive respirational devices such as endotracheal tubing, can cause a great deal of injury if accidently pulled out of or pushed into a patient. At the same time, assisting respiration of a patient, e.g., during sedation or even sleep, without need for such assistive respirational devices, is a preferred goal.

### SUMMARY OF THE INVENTION

Accordingly, it is an object of the present invention, to enhance respiration of an individual, especially when such individual might be unconscious.

It is also an object of the present invention to enhance ease of positioning and effectiveness of assistive devices for respiration, while maintaining as much comfort as possible for the patient.

It is a further object of the present invention, to enhance positioning and securing of conduits administering fluid to a patient or otherwise assisting in respiration or other medical treatment, to eliminate danger associated with use of such invasive devices.

Moreover, it is an object of the present invention to assist a patient in breathing while in bed, by effectively supporting the patient's head to maximize respiration and, at the same time, providing maximum comfort for the patient while bedridden.

These and other objects are attained by the present invention, which is directed to a device for thrusting a jaw of a patient forwardly and having at least one curved thrusting member both pivotally and translationally mounted to fit the patient's jaw and maintain the airway passages in the patient as open as possible. The present invention is also directed to a device for establishing a vacuum within a closed, rigid cage surrounding the neck of a patient to stent the upper airway open. A device of positioning and immobilizing a conduit administering respirational gases into a patient's body is also provided, especially for securing a nasally, orally or endotracheally inserted tubing into the patient.

The inventive devices provide secure, stabilization of the patient during respiration, minimizing discomfort of the patient while, at the same time, ensuring the requisite tubing cannot be inadvertently pulled out of or pushed too far into the patient.

The present invention also explicitly addresses and eliminates the above-noted problems of bedsores and head support, by providing therapeutic cushioning which can be used to support a patient for extended periods of time and re-distribute weight or pressure burden to surrounding areas of contact with the cushioning, thus reducing or totally eliminating the chance bedsores will occur.

More particularly, the present invention involves a series of cushions designed to decrease the amount of pressure placed on areas of the body's dependent anatomic regions by redistributing the weight burden to a larger surface surrounding the aforementioned dependent areas. The cushions are specifically designed to target the most dependent anatomic regions when the patient is placed in the supine and lateral positions.

The preferred embodiments are composed of single pieces of soft foam contoured into various shapes designed to redistribute pressure from a small, dependent surface area to a much greater surface area surrounding the dependent region. Alternatively, liquid, gas or gel-filled packets can comprise the body or the filling of the pillows.

The pillow designs are aimed at relieving pressure from the occiput and external occipital protruberance. Simultaneously, the design serves to relieve point pressure from the back of the user's neck while in the supine position.

The pillow is designed to contour to the posterior aspect of the patient's head and neck. Essentially, the pillow utilizes a circular design with differing radii of curvature and transition zones for differing anatomic areas.

In particular, the cushioning can be fashioned from any appropriately soft material such as foam, cotton or gelatinous substances. An optimal shape (as illustrated herein) includes a bi-concave disk having a furrowed notch or chamfer such that the round or nearly-round concavity is positioned substantially at the center of the disk-shaped cushioning and the furrow interconnects one edge of the cushioning to the concavity, to provide a receiving area and support for a patient's neck.

The inventive cushioning can be fashioned with opposite surfaces being mirror images of one another, i.e., each surface comprising an appropriately-dimensioned occipital concavity. Furrows or chamfering can be made in the same general areas of the cushioning on both sides thereof (e.g., at diametrically opposed ends to provide balancing) to intentionally weaken the outer rim of the cushioning and enhance collapsing upon receipt of a patient's neck. In the illustrated embodiment, a furrow or chamfer is provided only on one side of the disk-shaped cushioning. Furthermore, the furrows or chamfering can be made on opposite sides, both superiorly-inferiorly and anteriorly-posteriorly, to allow material strength to be maintained. Vertically-placed aeration holes can be fashioned through the pillow with a number of connections to the lateral edge.

Any combination of midline disk depth, diameter and angulation, as well as furrow dimensions, can be provided in accordance with the present invention. For extremely firm support, one side of the cushioning can remain unaltered to provide just a uni-concave disk. A uni-concave disk can have an electric or battery powered air pump connected to a circular aeration hose within an inferiorly-carved chamber. This chamber is contiguous with the vertical aeration holes. Such combination reduces scalp moisture and improves blood flow to the scalp, and also aids in controlling the patient's overall temperature as the airflow through the holes can be temperature-regulated. Cooler air temperatures can be utilized to decrease the metabolic demand of the patient's brain.

The present invention provides cushioning filled with a gel or gel-like material and designed to target the most dependent, i.e., vulnerable anatomical regions when a patient is in standard supine or lateral positions. The inventive cushioning is also especially suitable for supporting and securing medical apparatus against a patient to prevent skin breakdown, especially Continuous Positive Airway Pressure apparatus tubing for Neonates or infants.

The inner gel used in the cushioning may be composed of rubber polymers, differing amounts of tiny polystyrene foam beads, or a combination of both. Either inner combination can be covered by a soft, pliable material, such that the weight distribution will easily take place. Examples of material suitable for forming the outer cushioning include, but are not limited to, combinations of polyester, nylon, spandex and elastane, in ratios such as 85% polyester, 15% spandex, 82% polyester, 18% elastane, and 86% nylon, and 14% spandex. The pliable covering allows optimal distribution of the gel to the areas surrounding the most dependent body part.

Holes or windows are provided in several embodiments of the inventive cushioning to re-distribute the patient's weight or localized pressure. These openings are specifically designed to be placed underneath various bony protuberances, thereby allowing the surrounding soft tissue opportunity to re-distribute the patient's weight.

The inventive therapeutic cushioning can also be used to ameliorate effects of pre-existing bedsores on a patient.

### BRIEF DESCRIPTION OF THE DRAWINGS

The present invention will be described in greater detail with reference to the accompanying drawings in which

FIG. 1 illustrates a perspective view of a device for thrusting a jaw of a patient forwardly, in accordance with the present invention;

FIG. 1A illustrates a perspective view of a portion of an alternative embodiment of the jaw thrusting device illustrated in FIG. 1;

FIG. 2 illustrates a perspective view of a device for establishing a vacuum within a closed, rigid cage surrounding the neck of a patient to stent the upper airway open, in accordance with the present invention;

FIG. 3 illustrates an enlarged view, partially in section, of the encircled area 3 in FIG. 2;

FIG. 4 illustrates a perspective view of the device shown in FIG. 2 from an interior direction thereof;

FIG. 5 illustrates a perspective view of a device for positioning a conduit in accordance with the present invention;

FIG. 6 illustrates a perspective view of a device similar to the one shown in FIG. 5 and positioned about another part of a patient;

FIG. 7 illustrates a perspective view of a device for positioning a tube through the nose or mouth of a patient in accordance with the present invention ;

FIG. 8 illustrates a bottom plan view in the direction of arrows 8-8 shown in FIG. 7;

FIG. 9 illustrates a perspective view of a device for positioning a tube inserted into a patient through the nose, mouth or endotracheally in accordance with the present invention;

FIG. 10 illustrates a perspective view of a device for positioning and securing a tracheostomy tube in accordance with the present invention.

FIGS. 11A and 11B illustrate schematic plan views of one embodiment of the inventive cushioning, prior to assembly for supporting tubing, e.g., against the face of a patient;

FIG. 12 illustrates a front view of an embodiment of the inventive cushioning forming a cylinder;

FIG. 13 illustrates an end view of an embodiment of the inventive cushioning forming of FIG. 12 in the direction of arrow 13;

FIG. 14 illustrates an end view similar to FIG. 13 but showing an inventive cushioning having a larger diameter;

FIG. 15 illustrates a front view of the inventive cushioning shown in FIG. 14 in the direction of arrow 15;

FIG. 16 illustrates an end view of an embodiment of the inventive cushioning in the shape of a square;

FIG. 17 illustrates a front view of the inventive cushioning of FIG. 16 in the direction of arrow 17 and in the shape of a rectangular parallelepiped;

FIG. 18 illustrates a plan view of an embodiment of the inventive cushioning designed to support an ear and face of a patient; and

FIG. 19 illustrates a plan view of an embodiment of the inventive cushioning designed to support the occiput of a patient's cranium;

FIG. 20 illustrates a front perspective view from above of an alternative embodiment of the inventive cushioning in accordance with the present invention;

FIG. 21 illustrates a left side perspective view in the direction of arrow 21 in FIG. 20;

FIG. 22 illustrates a top plan view of the inventive cushioning shown in FIGS. 20 and 21;

FIG. 23 a bottom plan view of the inventive cushioning of FIGS. 20-22 and in the direction of arrow 23 in FIG. 20;

FIG. 24 illustrates a top plan view similar to FIG. 18 of an alternative embodiment of the inventive cushioning for supporting the lateral aspect of a patient's face;

FIG. 25 illustrates a front perspective view of yet a further alternative embodiment of the inventive cushioning for supporting a patient's occiput;

FIG. 26 illustrates a perspective view of the underside of the cushioning shown in FIG. 25;

FIG. 27 illustrates a front elevational view of a covering for the pillow comprising an interior space for fluid or gel;

FIG. 28 schematically illustrates positioning of pressure gauges on a head of a mannequin for measuring pressure upon various points of the inventive cushioning;

FIG. 29 schematically illustrates a side perspective view from above of a combination cushioning and bladder in accordance with the present invention;

FIG. 30 illustrates a sectional view in the direction of arrows 30-30 in FIG. 29;

FIG. 31 schematically illustrates a side perspective view from above of an alternative embodiment of the combination cushioning and bladder in accordance with the present invention; and

FIG. 32 illustrates a sectional view in the direction of arrows 32-32 in FIG. 31.

### DESCRIPTION OF THE PREFERRED EMBODIMENTS

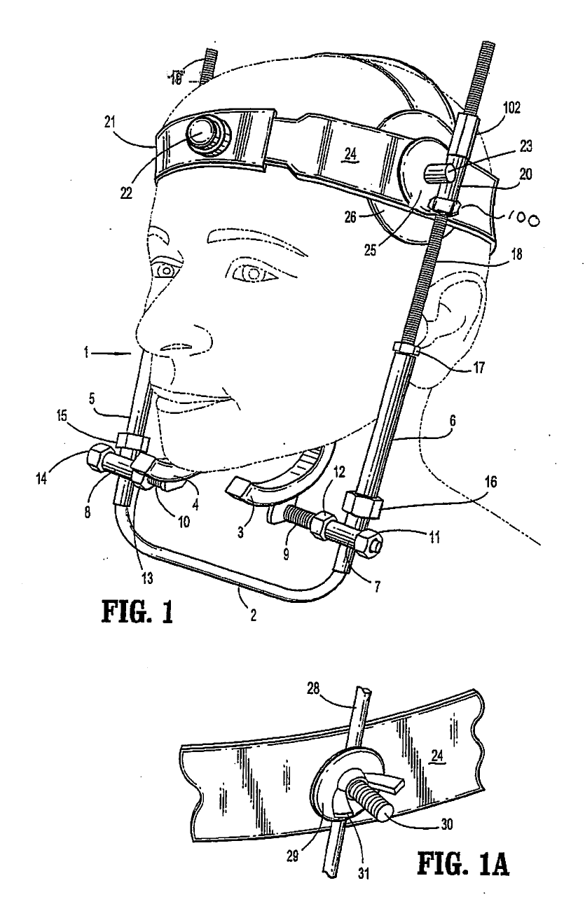

Referring to the drawings in the present application, FIG. 1 illustrates a device 1 for thrusting the jaw of a patient forwardly to permit the patient to breathe freely during sedation or sleeping. This device 1 can also be used to treat sleep apnea. The device 1 comprises a pair of curved thrusting members 3,4 designed to seat under the angle of the mandible as shown, and arranged to be both pivotally and translationally mounted to accommodate the size, shape and camber of a particular patient's jaw.

More particularly, a U-shaped member 2 has two legs 18, 19 in the form of screws and extending through respective cylinders 6,5. Jaw thrusting members 3,4 are pivotally and translationally mounted upon these respective cylinders 7, 8 respectively soldered on cylinders 6 and 5 (these cylinders are composed of metal). The jaw thrusting members 3,4 are each mounted upon screws 9 and 10 which are in turn positioned within respective cylinders 7 and 8 and secured in position and tilt by nuts 12, 11, 13 and 14. Therefore, the orientation of jaw thruster members 3 and 4, i.e., the tilting thereof and distance from one another, can be adjusted and then fixed to accommodate any shape of a jaw of a patient.

Additionally, the legs 18 and 19 of the U-shaped member is secured to a headband 24 having an adjustable circumference when positioned about a head of a patient by turning knob 22. Reference numeral 21 illustrates bucking mechanism to secure ends of the headband 24 together. Plastic discs 25, 26 are mounted upon headband 24 and, in turn, comprise metallic protrusions 23 to which copper cylinders 20 are soldered and through which legs 18 and 19 pass. Extension of these legs 18 and 19 through respective cylinders 20 is fixed by tightening nuts 100 and 102 against the respective cylinders 20. Additionally, position of legs 18 and 19 through respective cylinders 6 and 5 is fixed by tightening respective nuts 16, 17, 15, etc.

Therefore, the jaw thrusting device 1 can be adjusted to accommodate any size, shape or orientation of a patient's head and jaw, and specifically position thrusting members 3 and 4 to forwardly jut a patient's jaw to ensure unobstructed breathing, especially during sedation or sleeping. A patient's upper airway is therefore maintained patent by mobilizing the mandible and anteriorly pulling the base of the patient's tongue and soft tissue of the pharynx off the entrance to the trachea.

In an alternative construction shown in the partial view of FIG. 1A, plastic disc 29 secured to the headband 24 comprise an extending screw 30 upon which a wing nut 31 is tightened to secure leg 28 of the U-shaped member and which need not be threaded; in other features, this variant comprises the identical components shown in FIG. 1.

A spring mechanism (not illustrated) for biasing the U-shaped member 2 anteriorly, can be positioned, e.g., against headband 24 and legs 18, 19, or within or around protrusions 23.

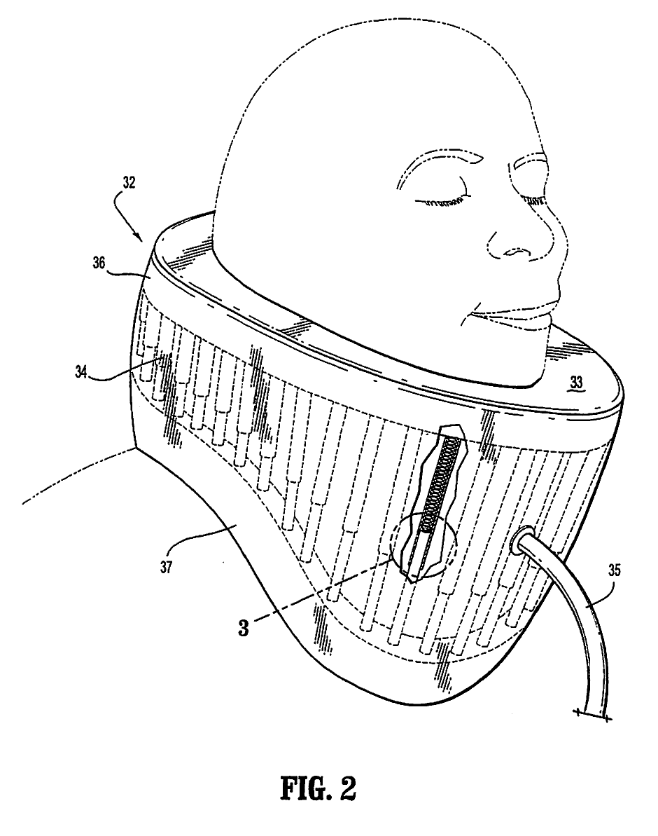

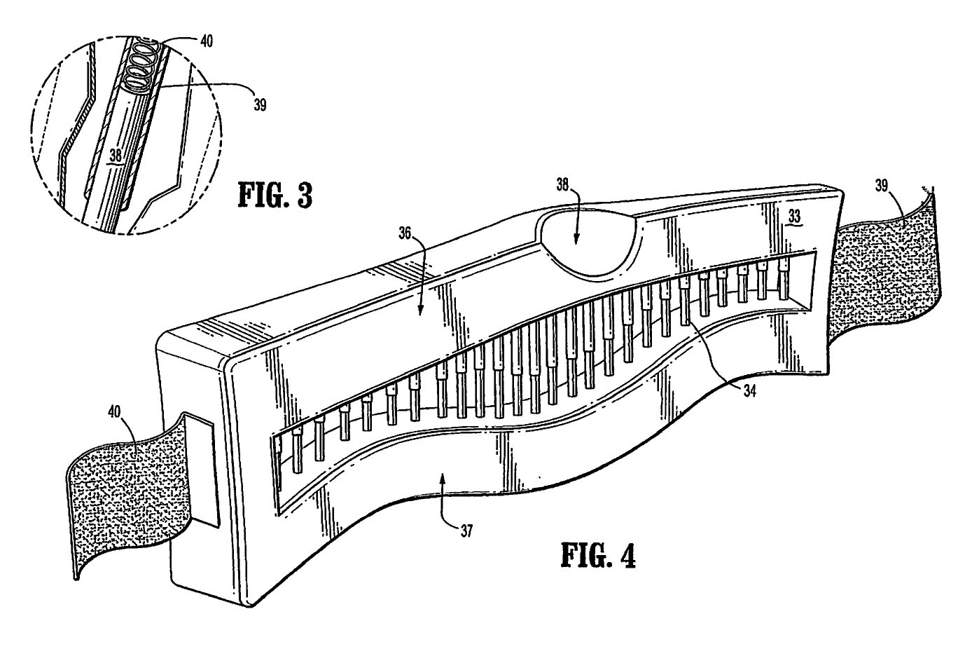

FIG. 2 illustrates a neck supporting device 32 when a vacuum is created around the neck through tube 35 to facilitate breathing by a patient. An outer flexible material made, e.g., of plastic (not shown) surrounds the device 32 and maintains the vacuum created through tube 35. More particularly, the device 32 is composed of a flexible membrane 33, e.g., a foam cushion shaped to encircle a patient's neck and an inner annular opening is cut through the foam cushion 33 to define upper 36 and lower 37 extending portions. As illustrated in FIG. 3, a series of spring-loaded 40 piston 38 and cylinder 39 arrangements, made of rigid plastic, are mounted across the opening between upper 36 and lower 37 extending portions.

The piston-cylinder arrangements 38, 39 are individually biased by the respective springs 40 from one another. The device 32 is secured around a patient's neck by velcro fasteners 39, 40 positioned at lateral ends thereof, with reference numeral 38 denoting a chin-rest cut in the upper extending member 36. The neck support device 32 shown in FIGS. 2 and 3 is extremely responsive to head and neck movement of a patient to comfortably accommodate the patient while a vacuum induced through tube 35 is maintained to stent the upper airway open.

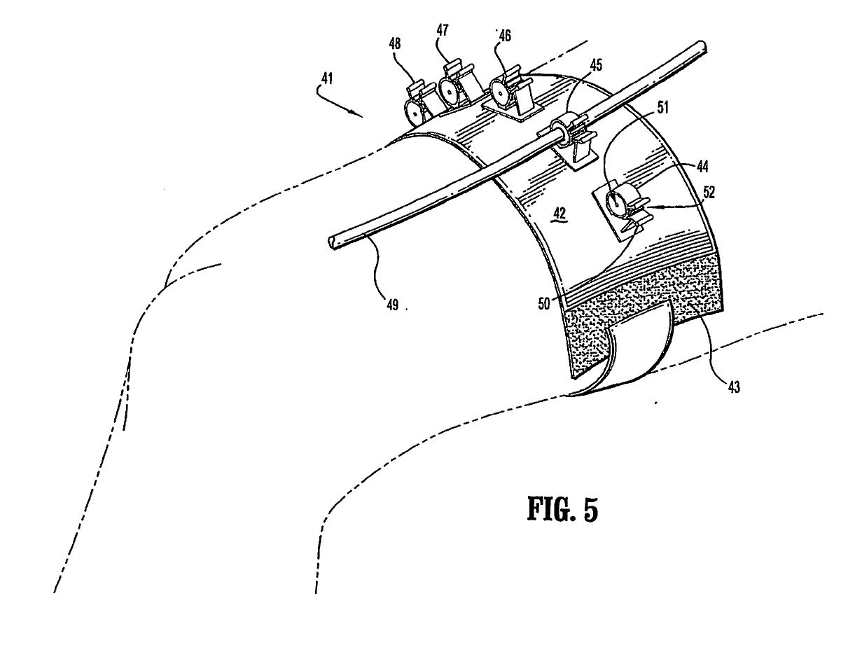

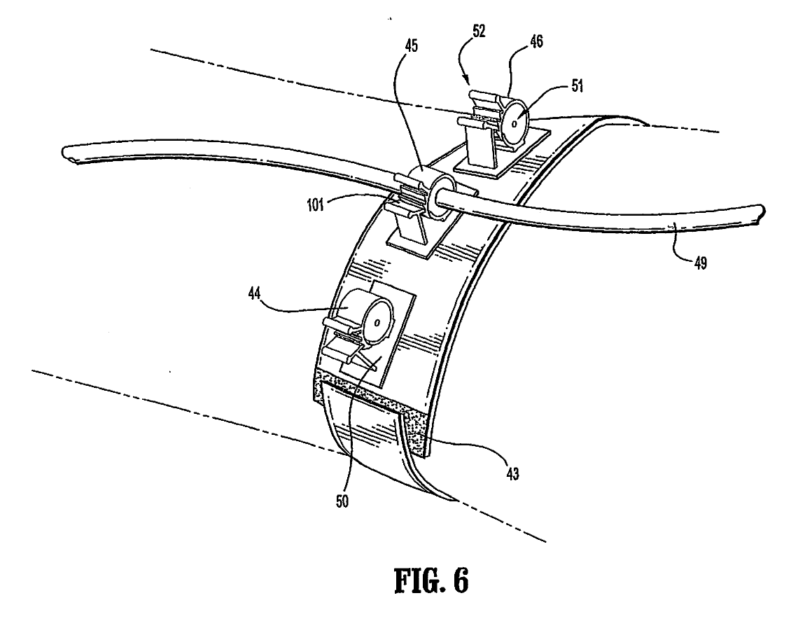

A device for 41 positioning and securing a conduit 49 for administering fluid to a patient, e.g., an intravenous tube, is illustrated in FIG. 5 as comprising a flexible belt 42 arranged to be secured around a patient's thigh by velcro fasteners 43 at opposite ends thereof. A series of ratchet clamps 44, 45, 46, 47, 48, etc. are mounted upon the belt 42 at respective bases 50. Each of these ratchet clamps 44, 45, 46, 47, 48, etc. comprises an opening 51 through which the tubing 49 is passed and a coupling snap 52 arranged to secure the tubing 49 passed through the respective clamp. As best seen in FIG. 6, the coupling snap 52 comprises a series of gradations 101 so that the tubing 49 can be securely retained by the coupled ratchet while, at the same time, flow of fluid through the tubing 49 remains unrestricted. FIG. 6 illustrates alternative positioning of a device similar to, but smaller than, device 41 around a forearm of a patient, instead of the patient's thigh as shown in FIG. 5.

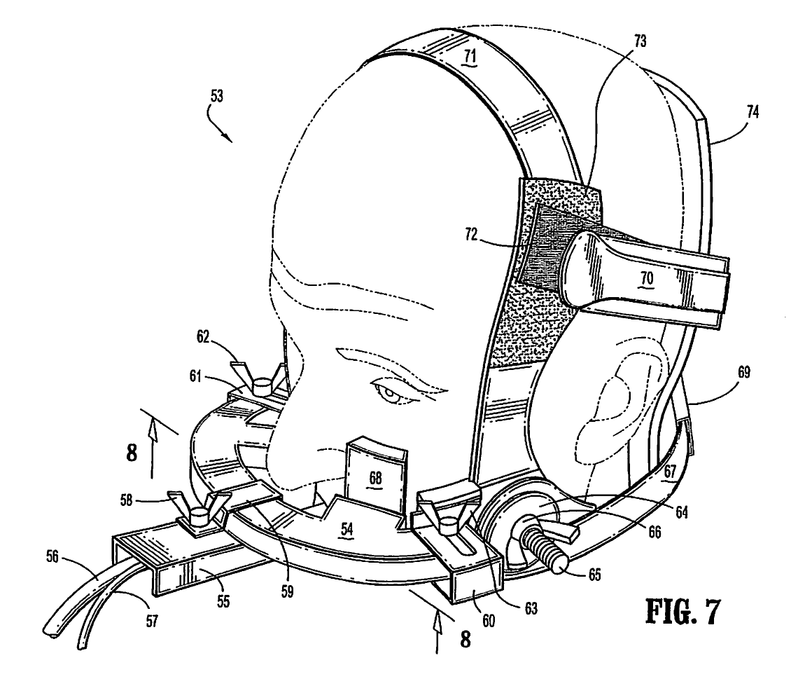

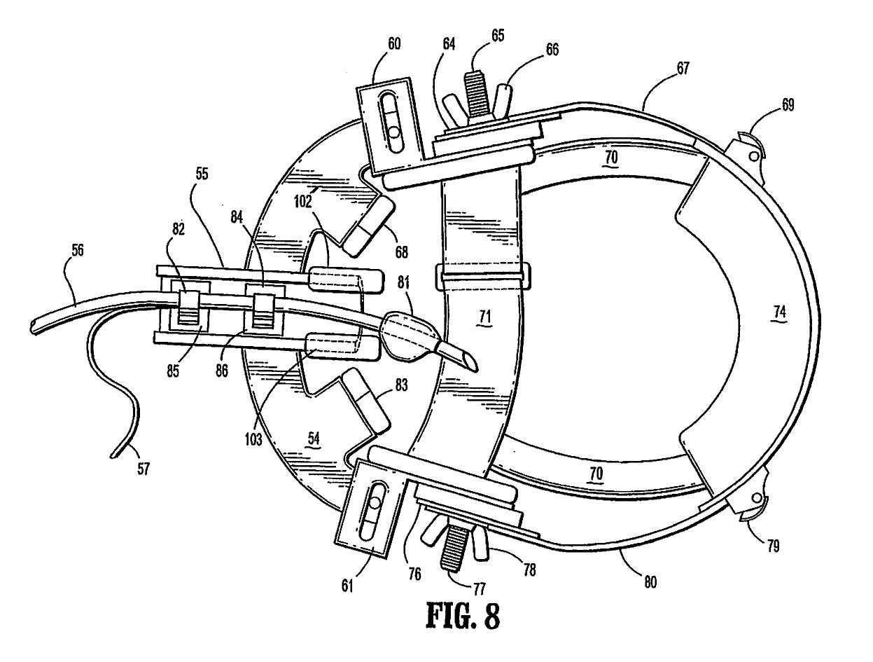

FIGS. 7 and 8 illustrate a device 53 for positioning and securing nasally or orally inserted tubes into a patient. In the embodiment illustrated in these two figures, an endotracheal breathing tube 56 and tube 57 carrying air to or from a cuffed balloon 81 are orally inserted through a patient's mouth and securely retained in position, so that the respective tubing 56, 57 cannot be pulled out or pressed in. A fiberglass platform 54 is adjustably secured to headgear to be positioned in front of a patient's face and retain an inverted U-shaped guide 55 secured thereto by a clamp 59 and wing nut 58. Ratchet clamps 82 and 84, similar to the ratchet clamps illustrated in the embodiments of FIGS. 5 and 6 supra, are secured to the underside of U-shaped guide 55 at respective bases 85, 86. Reference numerals 102′ and 103 denote bite pads of a patient's teeth, to prevent the patient from biting the tubing 56, 57 and interfering with respiration.

Fiberglass platform is cut with protrusions having cushions 68, 83 formed of soft foam or plastic material, at ends thereof and designed to seat against a patient's cheek. The headband itself comprises straps 71 and 70 designed to respectively wrap around the top and back of a patient's head and secure a plastic support 74 to the back of the patient's head as shown to provide comfort. These straps 71 and 70 are fastened to each other with velcro fasteners 72. An adjustable belt 67,80 is also provided to encircle the patient's neck and pass through a shield or guide 69 and be secured in position by ratcheting clamps or buckles 69 and 79.

The frontal platform 54 is secured to this belt 67, 80 through adjustable guides 60 and 61 positioned at opposite ends of the curved platform by tightening respective wing nuts 63. These clamps or guides 60 and 61 are, in turn, secured to the respective belt member 67 or 80 through respective screws 65, 77 mounted upon the belt members 67 or 80 and secured in place by the respective wing nuts 66, 78. Loosening the wing nuts 66, 78 allows the frontal platform 54 to be tilted with respect to belt members 67, 80 and allowing a change in angle of the frontal platform 54 with respect to the vertical, while loosening wing nuts 62, 63 allows the overall width of the device 53 between a patient's cheek to be adjusted, to thereby accommodate any size, shape or orientation of a patient's jaw.

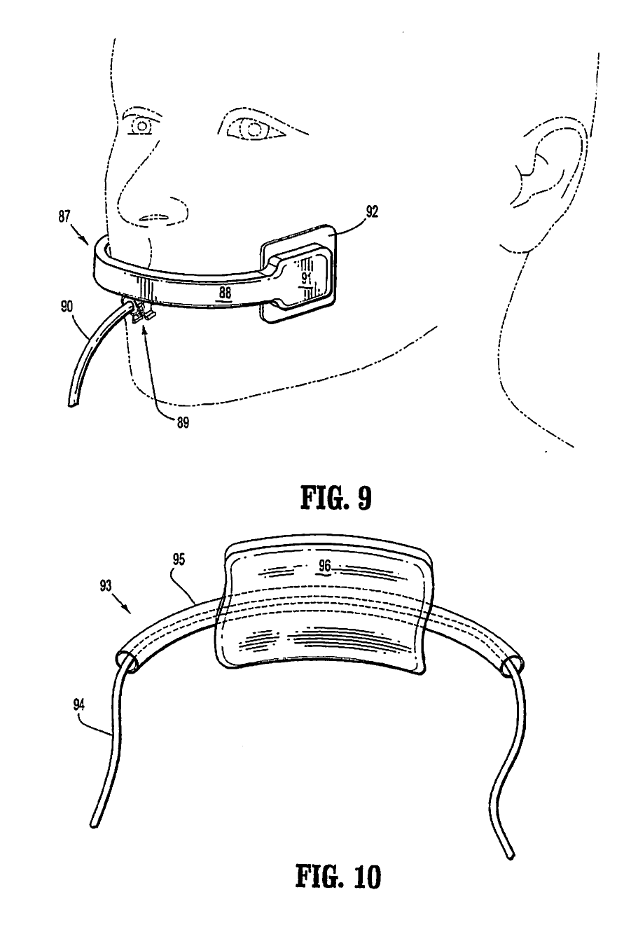

In FIG. 9, a device 87 for positioning an endotracheal tube 90 comprises a rigid, curved member 88 having adhesive pads 92 at opposite flared ends 91 thereof, with a ratchet clamp 89, similar to the ratchet clamps in the other embodiments described supra, being secured at a bottom end of the member 88 and arranged to position and secure the tubing 90 passing therethrough. FIG. 10 illustrates a padded device 96 attached to a tubular structure 93 composed of gel or foam through which a string 94 is passed and tied to the wings (not shown) of a tracheostomy tube. This device is designed to prevent skin breakdown on the neck of a patient while securing a tracheostomy tube.

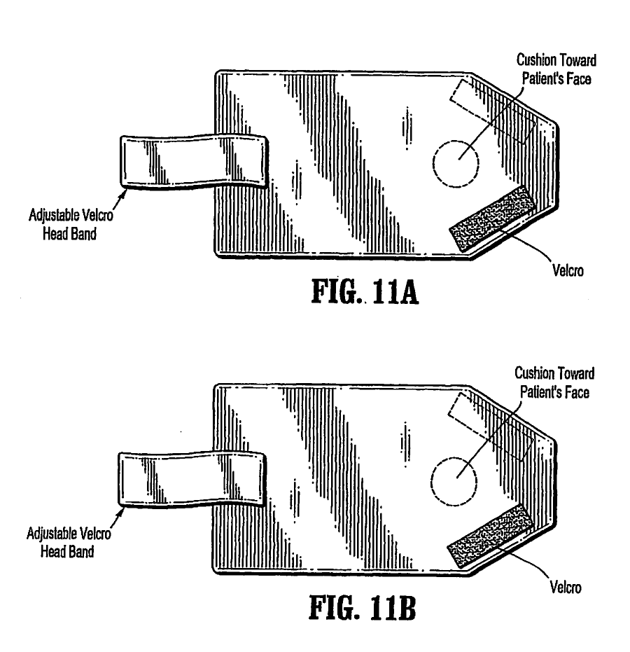

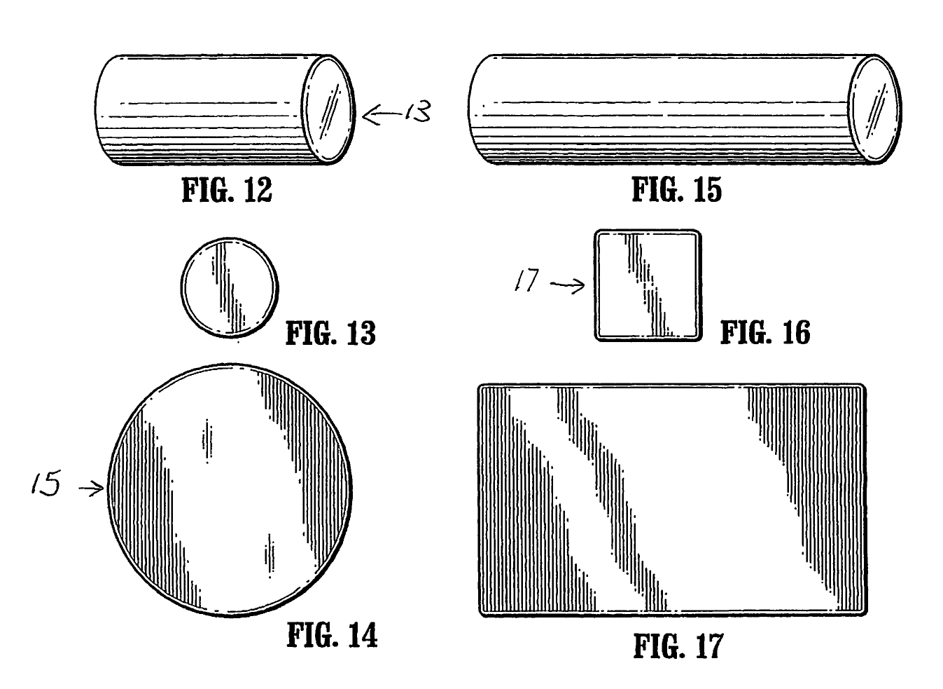

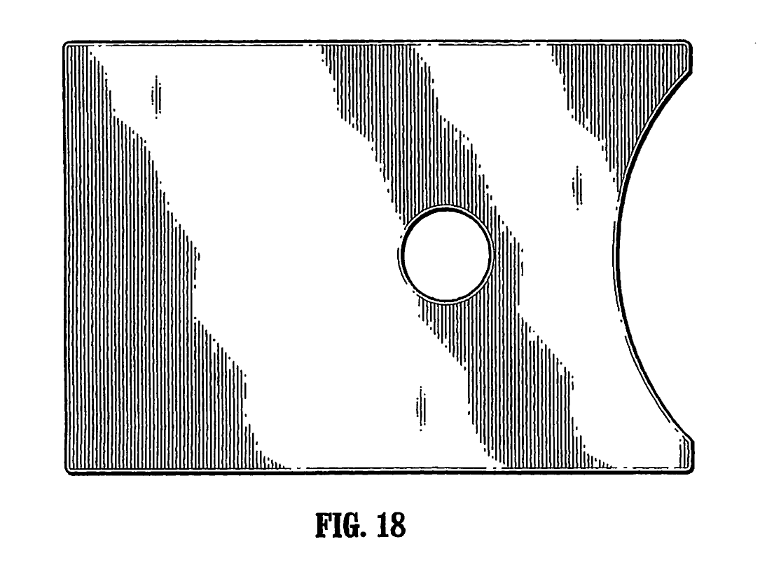

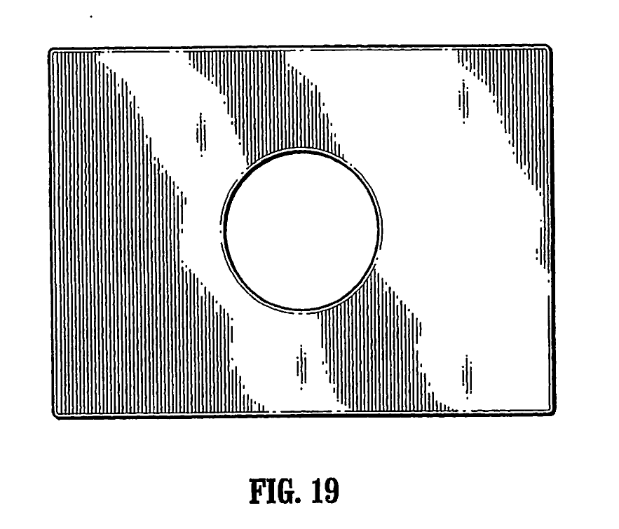

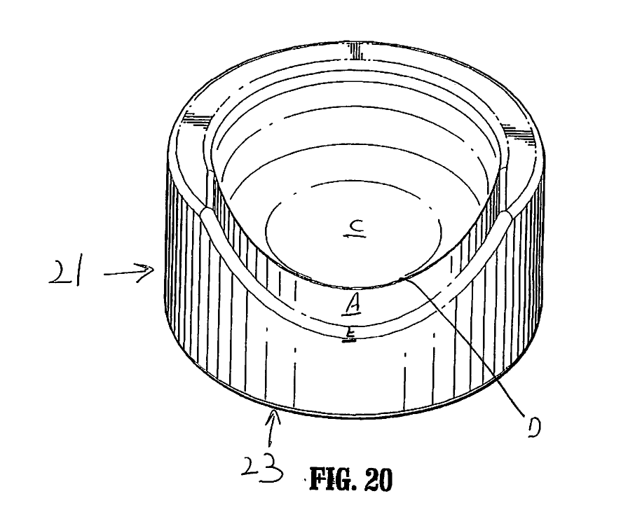

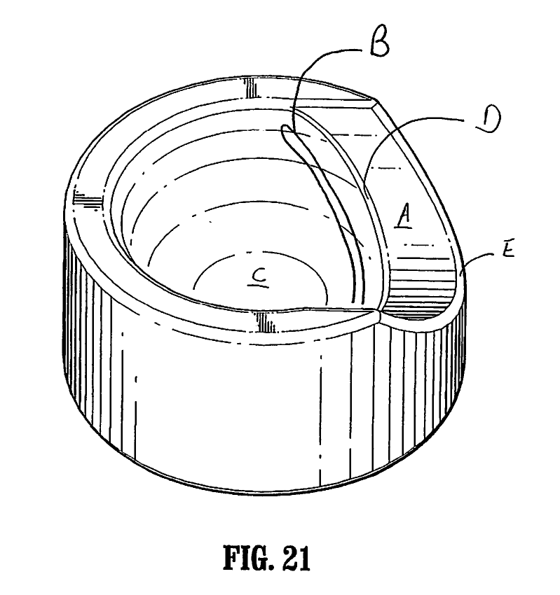

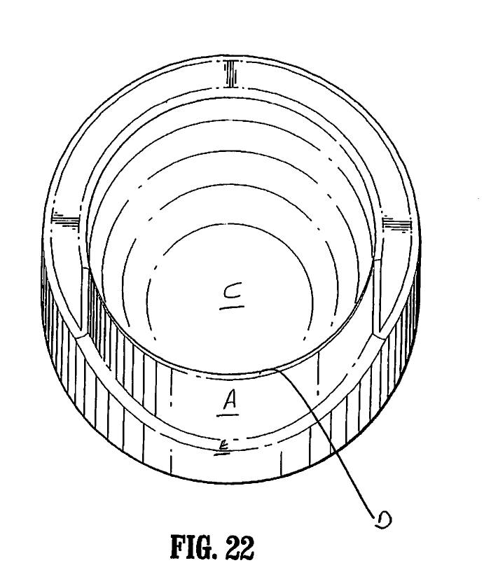

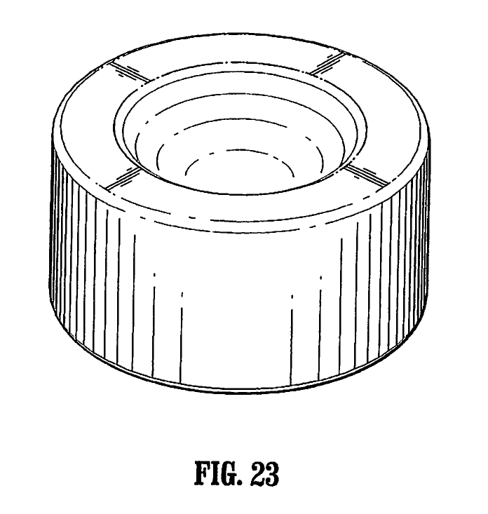

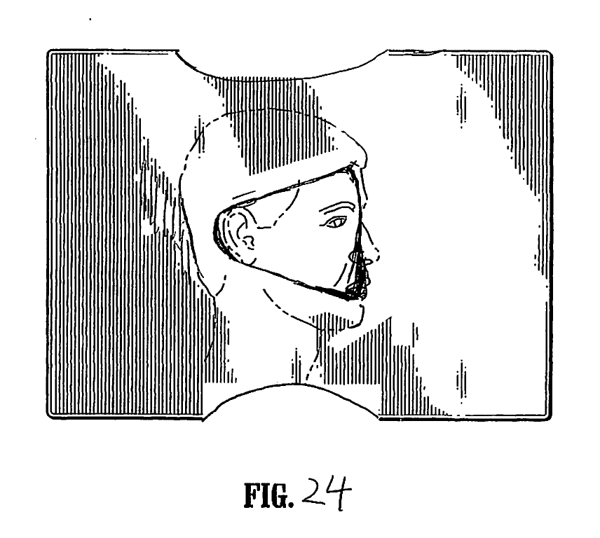

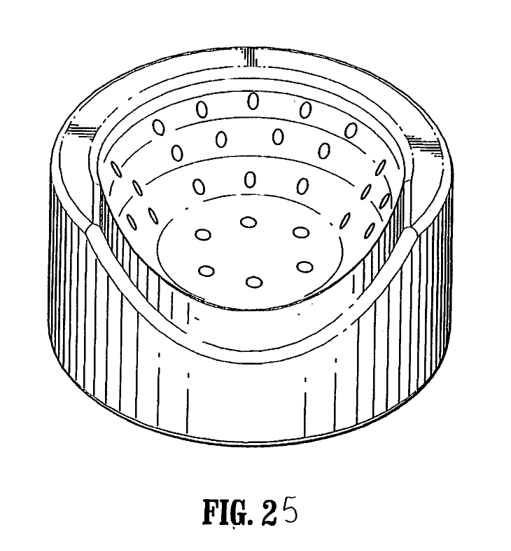

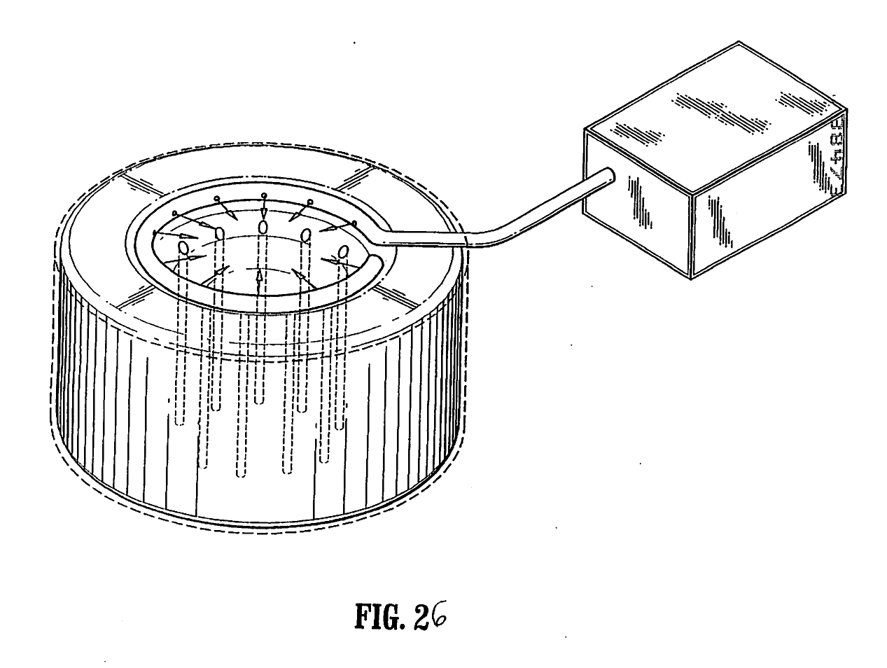

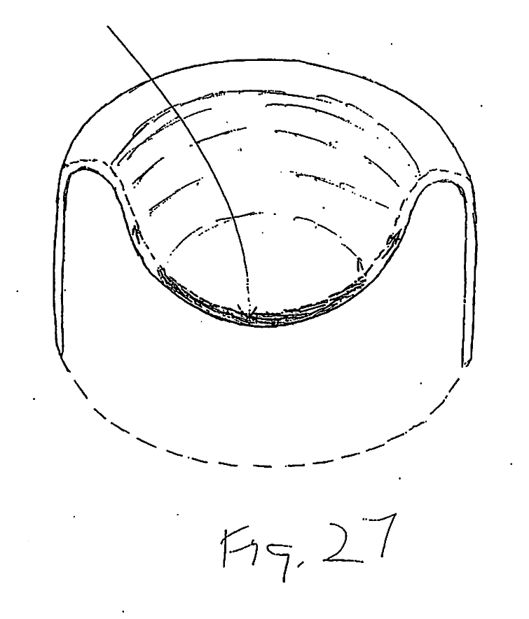

Regarding the therapeutic cushioning, the embodiment illustrated in FIGS. 11A and 11B is arranged to receive and encompass tubing therein and be hooked together, e.g., by velcro straps. Adjustable bands are provided. The embodiment shown in FIGS. 12-15, i.e., in the form of a cylinder, can be used to support and elevate the limbs of a laterally-placed patient, while the device shown in FIG. 18 is designed to receive the ear of the patient in an opening therein. Additionally, the device shown in FIG. 19 is also designed to receive, support and cushion the occiput of a patient.

Referring to the embodiment of the inventive cushioning illustrated in FIGS. 20-23 of the present application, the inventive cushioning can be fashioned in a concave or bi-concave shape, with each concavity arranged to receive, support and cushion the occiput of a supinely-positioned patient. The concavity can vary in diameter, depth and angulation, and increases surface area of a patient's head resting against the cushion and upon which pressure of weight of the patient's head is distributed. Additionally, a portion of the rim of the cushioning defining the concavity is chamfered, e.g., on one side as illustrated, with sloping convexity for receiving, supporting and cushioning the neck of the supinely-positioned patient while the patient's occiput is simultaneously received in the middle concavity of the cushioning. Such sloping convexity of the rim of the cushioning can also vary in width, depth, central and peripheral angulation. The material forming the cushioning can be composed of foam, cotton, polyester or any other suitably soft material that will comfortably accept the occiput and neck of the patient and conform to the shape of the patient's head and neck.

Additionally, a number of slit-like incisions can be made in the material to soften the same so the neck and occiput will be accepted more conformingly. Furthermore, the material forming the middle of the cushioning can be different from material forming the outer rim, in accordance with the present invention.

As illustrated herein, the opposite “underside” of the disk-shaped cushioning can also be fashioned with an occipital-receiving concavity and/or cervical chamfering of differing widths, depths and angulations such that a patient can vary position of the various pressure points contacting the cushioning, as required.

In the illustrated embodiment in FIGS. 20-23, the cervical support (A) has the smallest central radius of curvature. Its contoured groove works to increase the contact surface area, reduce pressure points, support the neck, optimize the patency of the upper airway and minimize pressure at the atlanto-occiptal joint. It is devised to avoid hyperextension or hyperflexion of the user's upper airway.

The cervical support can also be designed to accept an artificial cervical collar (in the form of a commercially available rigid, semi-rigid, or soft cervical collar for patients with injuries to the neck region). In such a case, the cervical support (A) has a greater radius of curvature, a wider lateral groove, and a deeper concavity than a cervical support (A) accepting a native neck.

The occipital groove (B) is designed to accept the external occipital protruberance of the skull and diffuse the contact surface area. This area has the largest radius of curvature, as the external occipital protruberance bulges outwardly from the posterior aspect of the skull.

The occipital cradle (C) is designed to accept the occiput and diffuse the contact surface area. The cradle consists of a deep groove with varying radii of curvature, reflecting the natural formation of the human skull. The human skull is neither an exact sphere nor the same shape in each person. The contour and material of the occipital cradle reflect both of these facts. The cradle is designed with soft, adaptable foam to increase the contact surface area of each individual user's head. It is a high-rising design, aimed at utilizing the lateral and postero-lateral portions of the user's head to accept some of the pressure burden while lying supine.

Transition zones (D) and (E) between the aforementioned sections are smooth and designed to mimic the natural contour of the neck and head.

An optional fluid or gel-filled insert can be placed over the pillow to improve surface contact for patients with a small or irregularly-shaped head. The insert fits over the lateral edges of the pillow, and a flexible, double concave, air-tight and air deficient fluid or gel-filled portion sits just inside the occipital cradle. (C). The fluid can be warmed or cooled, if desired, to optimize the blood flow to the scalp, or aid in fever reduction. For patient's able to move their head, this insert will significantly reduce the friction forces on the occiput by providing a contact surface with the head that will substantially accompany the head during lateral, superior and inferior movement.

This contact surface are will, in turn, glide smoothly along the fluid or gel medium on top of the substantially immobile inferior contact surface area adjacent to the pillow itself. This decrease in frictional force on the occiput also serves to significantly reduce pressure ulceration to the back of the head.

The inventive therapeutic cushioning illustrated in FIGS. 20-23 can be prepared with the following dimensions to accommodate the following shapes of the occiput of an average adult patient:

Top of Pillow

| Height from base of nadir | Approximate diameter of |
|---|---|
| of occipital cutout (inches) | occipital cutout (inches) |
| 0.5 | 2.25 |
| 1.0 | 3.0 |
| 1.5 | 4.5 |
| 2.0 | 5.5 |
| 2.75 | 8 |

Bottom of Pillow

| Height from base of nadir | Approximate diameter of |
|---|---|
| of occipital cutout (inches) | occipital cutout (inches) |
| 0.5 | 2.25 |
| 1.0 | 3.0 |
| 1.5 | 4.5 |
| 1.75 | 6.5 |

Pillows can be formed in an alternative size to accommodate an adult, child or infant head. The smallest pillows are contoured with the occipital cradle (C) having the smallest radius of curvature, reflecting the natural shape of the infant's head. The cervical support (A) and the occipital cradle (C) of the infant pillow will reflect the larger proportional size of the infant occiput relative to total body surface area. The child-size pillow will have dimensions and radii of curvature between the infant and adult pillows to reflect the natural growth and dimensionsal changes of the human skull and cervical anatomy.

The pillow's foam is anti-bacterial, non-allergenic and flame-retardant. Each pillow will be encased in a similarly-shaped, pliable, fluid-proof, anti-bacterial cover that is easily launderable and possesses multiple aeration holes to prevent moisture accumulation and improve air flow to the skin. These features contribute to the reduction of skin breakdown and decubitus ulcer formation. In short, the pillow is a semi-rigid orthopedic device used to support the head and neck while diffusing the pressure on the most dependent anatomic areas. The design increases the blood flow to the occiput relative to traditional pillows, and decreases the risk of skin breakdown and ulcer formation in patients who are not able to move their head (patients who are immobile due to illness, accident or injury). The optional fluid or gel-filled insert provides additional advantages of temperature control, reduced friction and improved surface area contact.

The pillows may also be created with a variety of other materials. They may be layered with an inner material other than foam. These may include (but are not limited to) an inner gel consisting of rubber polymers, differing amounts of tiny polystyrene foam beads, or a combination of both. Either inner combination can be covered by a soft, pliable material, such that a variable weight distribution will easily occur when the patient places the occiput on the pillow. Examples of outer material include, but are not limited to, combinations of polyester, nylon, spandex and elastane, in ratios such as 85% polyester, 15% spandex, 82% polyester, 18% elastane, and 86% nylon, and 14% nylon. The pliable covering allows optimal distribution of the gel to the areas surrounding the most dependent body part.

Minimizing point pressure on a small surface areas of the back of the head is a major goal of the therapeutic cushioning or pillows. To prove the pressure is re-distributed across a greater surface areas of the head and, therefore, reduced in the occiput, a pressure gauge system is provided and comprises a snugly-fitting rubber cap which is placed on a patient's head in similar manner to a swimming cap. Attached to the cap are multiple strategically-placed, flat pressure gauges. Each gauge is connected with tiny wires to a computerized central processing box in which digital pressure readings from each gauge are process and displayed.

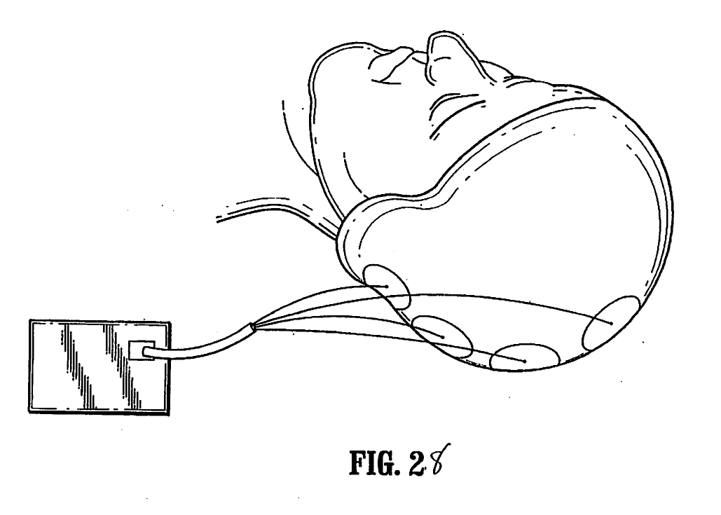

By utilizing such a device, each individual can document pressure changes on varying scalp surfaces when lying on a flat pillow and/or the inventive therapeutic cushioning. Since the pressure gauge system conforms to the surface of an individual's scalp, and contains flat pressure gauges, realistic measurement of angular forces can be obtained. Wires can be run through the aeration holes or channels of the pillow to avoid exposure. As shown in FIG. 28, the pressure gauge system can be incorporated upon a mannequin head which is removable to allow the wires to exit therefrom.

For cushions other than pillows, either the foam, gel or bead design can be fashioned into square, oblong or round cushions with holes or windows fashioned into them to re-distribute the patient's weight. These areas are specifically designed to be placed underneath various bony protuberances, thereby allowing the surrounding soft tissue an opportunity to re-distribute the weight.

In addition, this technology has been applied for use in securing Continuous Positive Airway Pressure apparatus tubing for Neonates or infants.

The present invention is composed of a series of pillows and cushions designed to redistribute weight, reduce point-pressure, and preserve or maintain skin integrity. Conventional practice generally involves providing treatment once bedsores have started developing. However, by using these products on those patients identified by the Braden Scale to be at risk for developing skin breakdown in institutional settings or for those who are bed-fast at home, considerable savings in pain, time and money could result.

As bedsores tend to develop around bony prominences, use of the various cushion products could potentially reduce or eliminate the likelihood that pressure sores will develop. These products could be used in concert with traditional methodology of turning patients every 2 hours and providing adequate nutrition and hydration.

The pillow and cushion designs are viable, cost effective solutions to prevent skin breakdown in bed-bound patients, both at home and in institutional settings.

The largest benefit of these products is aimed at patients with limited/non-existent mobility in institutional and home settings. Over 1 million persons or 10% of hospital patients are afflicted with bedsores in US hospitals every year, according to the National Decubitus Foundation. Research has revealed that all patients regardless of age are at risk for skin breakdown if they are confined to bed for extended periods. Nursing home patients are particularly at risk, given their overall health status. Risk factors for pressure sores include advancing age, being unable to position oneself, poor or decreased nutrition, moisture, decreased sensory perception and being bedfast or chairfast.

Healthcare costs are rising and there appears to be a trend towards prevention and proactive medicine rather than reactive medicine. By targeting specific populations such as Neo-Natal Intensive Care Unit babies, the elderly in Nursing homes, those patients in Rehabilitation facilities, patients in Intensive Care Units and home care patients, acceptance of these products is almost certain.

By providing various combinations of pillows and cushions, the financial, emotional and physiological savings will be extraordinary.

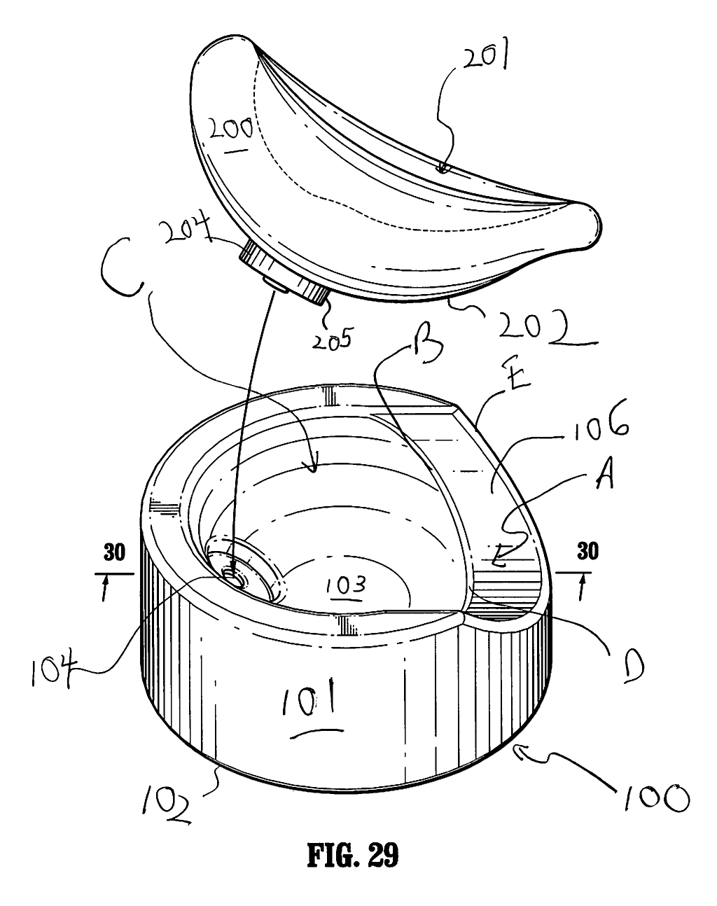

In particular, the present invention is directed to a series of cushions and bladders in combination as illustrated, e.g., in FIGS. 29-32 designed to decrease amount of pressure placed on areas of the body's dependent anatomic regions by redistributing weight burden of the head region of a patient to a larger surface surrounding the dependent areas of a patient's head. The cushion/bladder combination is specifically designed to target the most dependent anatomic regions of the head when a patient is placed in supine and lateral position.

The cushioning or pillow 100 itself is designed to contour to the posterior aspect of the patient's head and neck. Essentially, the pillow utilizes a circular design with differing radii of curvature and transition zones for differing anatomic areas. The cervical support area (A) having the smallest central radius of curvature is a contoured groove working to increase contact surface area, reduce pressure points, support the neck, optimize patency of the patient's upper airway and minimize pressure at the atlanto-occiptal joint. It is devised to avoid hyperextension or hyperflexion of the patient's upper airway.

The cervical support can also be designed to accept an artificial cervical collar in the form of a commercially available rigid sem-rigid or soft cervical collar for patients with injuries to the neck region. Occipital groove (B) is designed to accept the external occipital protuberance of the skull and diffuse the contact surface area. This area has the largest radius of curvature as the external occipital protuberance bulges outwardly form the posterior aspect of the skull.

The occipital cradle (C) is designed to accept the occiput of a patient and diffuse the contact surface area, consisting of a deep groove with varying radii of curvature, reflecting the natural formation of the human skull and being a high-rising design aimed at utilizing the lateral and postero-lateral portions of a patient's head to accept some of the pressure burden while the patient is lying supine.

The transitional zones (D) and (E) between the above-noted sections are smooth and designed to mimic the natural contour of the patient's head and neck. The foam constituting the cushioning 100 itself is anti-bacterial, non-allergenic and flame retardant. Each cushioning is encased in a similarly-shaped, pliable, fluid-proof, anti-bacterial cover that can be easily laundered. Furthermore, each pillow or cushioning possesses multiple aeration holes to prevent moisture accumulation and improve air flow to the skin. These features contribute to reduction of skin breakdown and decubitus ulcer formation.

Additionally, the cushioning can be constructed of material other than foam, such as, but not limited to, an inner gel consisting of rubber polymers, differing amounts of tiny polystyrene foam beads or a combination of both. Either inner combination can be covered by a soft pliable material, such that a variable weight distribution will easily occur when the patient places the occiput on the pillow. Examples of other material include, but are not limited to, combinations of polyester, nylon, spandex and elastane, in ratios such as 85% polyester and 15% spandex or 82% polyester and 18% elastane. For cushions other than pillows, either the foam , gel or bead design can be fashioned into square, oblong or round cushions with holes or windows fashioned into the same to distribute the patient's weight. These areas are especially designed to be placed underneath various bony protuberances, thereby allowing the surrounding soft tissue an opportunity to re-distribute the weight.

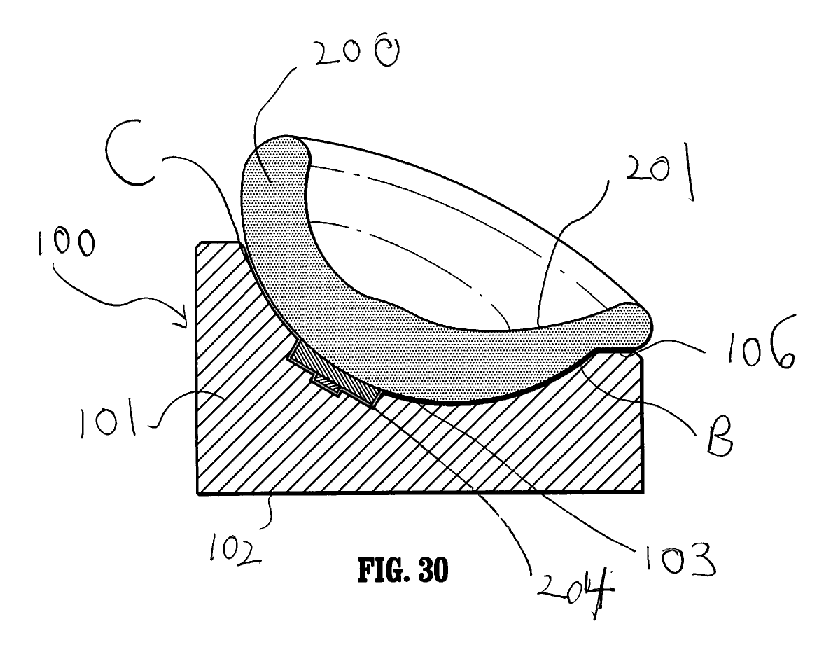

As shown in FIGS. 29 and 30, the cushioning 100 has a cylindrical foam base 101 (8 or 9 inches in diameter and 5 inches in height for adults) flat on its bottom 102 to contact the bed surface. Additionally, the “head” surface has one edge 106 “carved” to create a cervical support for the user, serving to align the upper airway as well as provide a surface to support a portion of the gel-filled pack or bladder 200 that provides increased surface area to reduce point-pressure and decrease shearing forces on a patient's neck.

The deepest portion 103 of the cushioning headrest 100 has a recess or opening 104 to accommodate a cap or filling valve 204 of the bladder 200. This additional cut 104 is shaped to complementary conform to the shape of the cap 204. The sac portion of the bladder 200 can be fabricated from silicone or latex rubber, or similar sturdy yet pliable material. The bladder 200 is preferably formed from non-latex silicone rubber and may be fabricated by dip-molding or spin-molding. The shape itself of the bladder 200 is concave on top 201 to conform with the occipital head aspect of a patient. One-quarter to one-third of the circumference of the bladder 200 is extended outwardly from both top 201 and bottom 202 layers for approximately 6 inches in an adult bladder and 4 inches in a pediatric bladder. This portion is also concave from the superior aspect in both the “neck” and “bed” portions.

The bladder is filled approximately ⅓ to capacity with gel, then de-aired and sealed. In a preferred embodiment, the filling/sealing cap 204 is seated in the deepest recess 104 of the cushioning 100. Such cap 204 is a polyvinyl chloride screw/clamp commercially available as a plug. The ring portion 205 of the cap 204 is placed outside an extension of a silicone cover which is then draped around the bladder 200 after the bladder 200 is filled. The screw portion is placed on an inside of the same extension of the silicone cover which is firmly secured between the two portions.

The concept of the gel-filled bladder 200 contained within the cushioning 100 can also be contoured to other parts of the human anatomy such as knees, elbows or heels. Any suitable therapeutic gel, e.g., a hot/cold pack gel, can be filled into the bladder 200.

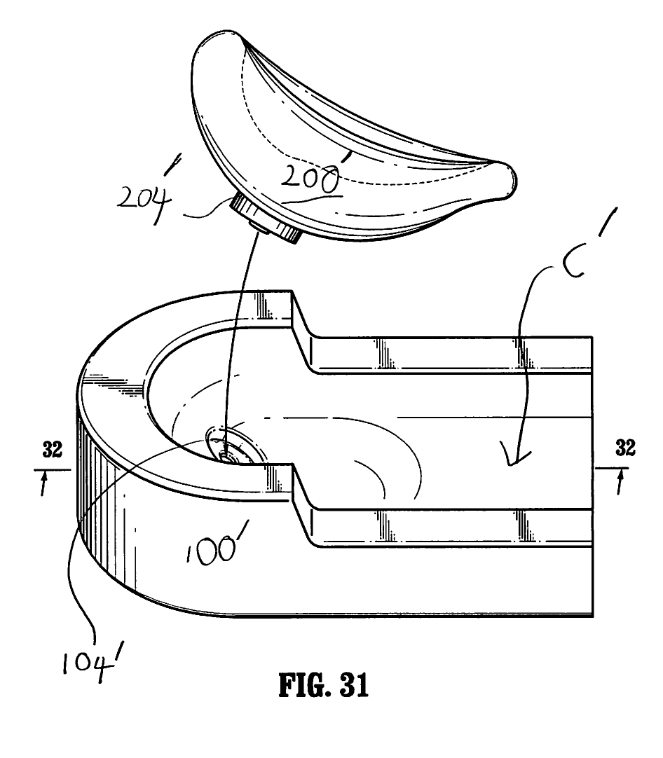

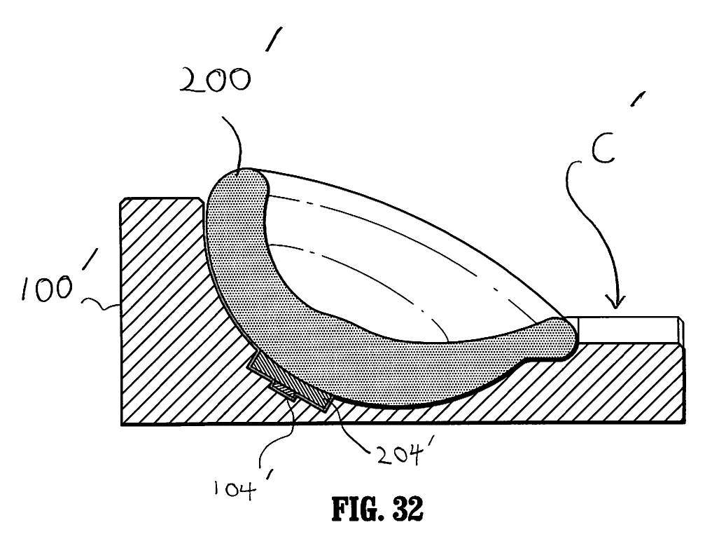

While FIGS. 29 and 30 illustrate a substantially cylindrical cushioning 100 to receive gel-filled bladder 200, FIGS. 31 and 32 illustrate and alternative embodiment where cushioning 100′ is oblong shaped together with “occipital” cradle (C′) but with opening 104′ still positioned to conform with end cap 204′ of bladder 200′. This embodiment is useful, e.g., to support an elbow or heel of a patient.

The preceding description of the present invention is merely exemplary and not intended to limit the scope thereof in any way.

What is claimed is:

1. Therapeutic cushioning structured and arranged to support a head and neck of a patient, comprising
a closed, bowl-shaped, curved concavity having a curved bottom surface arranged on at least one side thereof to curve underneath and receive and support an occiput of the patient's head on the bottom surface thereof, and
a furrow or chamfer extending over and along a top edge of the cushioning and leading to the concavity, and being arranged to receive and support a neck of the patient thereon,
wherein the concavity comprises a varying radius of curvature with
a cervical support section having a smallest of said varying radius of curvature, followed by
an occipital groove having a largest of said varying radius of curvature,
an occipital cradle, and
transition zones between the cervical support section and occipital groove and cradle.

2. Therapeutic cushioning of claim 1 substantially in the shape of a disk.

3. Therapeutic cushioning structured and arranged to support a head and neck of a patient, comprising
two closed, bowl-shaped, curved concavities, each having a curved bottom surface and positioned on opposite sides of the cushioning from one another,
with at least one of said concavities arranged to curve underneath and receive and support an occiput of the patient's head on the bottom surface thereof, and
a furrow or chamfer extending over and along a top edge of the cushioning and leading to said at least one concavity, and being arranged to receive and support a neck of the patient thereon.

4. Therapeutic cushioning of claim 5, wherein the furrow or chamfer is located on only one side thereof.

5. Therapeutic cushioning structured and arranged to support a head and neck of a patient, comprising
a closed, bowl-shaped, curved concavity having a curved bottom surface arranged on at least one side thereof to curve underneath and receive and support an occiput of the patient's head on the bottom surface thereof,
a furrow or chamfer extending over and along a top edge of the cushioning and leading to the concavity, and being arranged to receive and support a neck of the patient thereon, and
a groove situated along an inner circumferential surface of said concavity and arranged to specifically receive the occiput of the patient thereon.

6. Therapeutic cushioning of claim 5, additionally comprising a series of channels interconnecting the inner circumferential surface with an outer edge surface of said cushioning.

7. Therapeutic cushioning structured and arranged to support a head and neck of a patient, comprising
a closed, bowl-shaped, curved concavity having a curved bottom surface arranged on at least one side thereof to curve underneath and receive and support an occiput of the patient's head on the bottom surface thereof,
a furrow or chamfer extending over and along a top edge of cushioning and leading to the concavity, and being arranged to receive and support a neck of the patient thereon, and
a series of channels interconnecting said concavity with an outer edge surface of said cushioning.

8. Therapeutic cushioning of claim 7, additionally comprising a groove cut along an underside of said cushioning and arranged to receive tubing communicating with said channels.

9. Therapeutic cushioning claim 8, additionally comprising a second groove cut along an underside of said cushioning and arranged to receive tubing communicating with said channels.

10. Therapeutic cushioning of claim 9, additionally comprising a separate gel-pack arranged to be received in said concavity and heated or cooled.

11. Therapeutic cushioning of claim 1, additionally comprising a separate gel-filled bladder arranged to be received in said concavity and heated or cooled.

12. The therapeutic cushioning of claim 1, additionally comprising a series of channels interconnecting said concavity with an outer edge surface of said cushioning.

13. Therapeutic cushioning of claim 3, additionally comprising a separate gel-filled bladder arranged to be received in said concavity and heated or cooled.

14. The therapeutic cushioning of claim 3, additionally comprising a series of channels interconnecting said concavity with an outer edge surface of said cushioning.

15. Therapeutic cushioning of claim 5, additionally comprising a separate gel-filled bladder arranged to be received in said concavity and heated or cooled.

16. The therapeutic cushioning of claim 5, additionally comprising a series of channels interconnecting said concavity with an outer edge surface of said cushioning.

17. Therapeutic cushioning of claim 7, additionally comprising a separate gel-filled bladder arranged to be received in said concavity and heated or cooled.

18. The therapeutic cushioning of claim 7, additionally comprising a series of channels interconnecting said concavity with an outer edge surface of said cushioning.

19. The therapeutic cushioning of claim 11, additionally comprising a recess extending through a bottom of the cushioning and arranged to receiving a filling cap of the gel-filled bladder.

20. The therapeutic cushioning of claim 13, additionally comprising a recess extending through a bottom of the cushioning and arranged to receiving a filling cap of the gel-filled bladder.

21. The therapeutic cushioning of claim 15, additionally comprising a recess extending through a bottom of the cushioning and arranged to receiving a filling cap of the gel-filled bladder.

22. The therapeutic cushioning of claim 17, additionally comprising a recess extending through a bottom of the cushioning and arranged to receiving a filling cap of the gel-filled bladder.

23. The therapeutic cushioning of claim 1 being oblong-shaped and having an occipital cradle of oblong shape.

24. The therapeutic cushioning of claim 3 being oblong-shaped and having an occipital cradle of oblong shape.

25. The therapeutic cushioning of claim 5 being oblong-shaped and having an occipital cradle of oblong shape.

27. The therapeutic cushioning of claim 7 being oblong-shaped and having an occipital cradle of oblong shape.
